(19) United States
(12) Patent Application Publication
(10) Pub. No.: US 2018/0167200 A1
(43) Pub. Date: Jun. 14, 2018

(54) OBTAINING A MEDICAL RECORD STORED ON A BLOCKCHAIN FROM A WEARABLE DEVICE

(71) Applicant: Wal-Mart Stores, Inc., Bentonville, AR (US)

(72) Inventors: Donald R. High, Noel, MO (US); Bruce Walter Wilkinson, Rogers, AR (US); Todd Mattingly, Bentonville, AR (US); Robert Cantrell, Herndon, VA (US); John J. O'Brien, V, Farmington, AR (US); Brian Gerard McHale, Oldham (GB); Joseph Jurich, JR., Molino, FL (US)

(21) Appl. No.: 15/840,589

(22) Filed: Dec. 13, 2017

Related U.S. Application Data
(60) Provisional application No. 62/433,956, filed on Dec. 14, 2016.

(51) Int. Cl. H04L 9/08; G16H 10/60; H04L 9/06; G06F 21/62; A61B 5/00; H04L 9/32; G06K 19/07

(52) U.S. Cl. CPC H04L 9/08; G16H 10/60; H04L 9/0637; H04L 67/104; A61B 5/6824; H04L 9/3231; G06K 19/0723; G06F 21/62

(57) ABSTRACT

A method for obtaining a medical record of a patient that is unable to communicate, wherein the medical record of the patient is stored on a blockchain, is provided, including receiving an encrypted private key and a public key associated with the patient stored on a wearable device of the patient, in response to a scanning of the wearable device of the patient at a scene of an emergency, wherein the encrypted private key is decrypted by a biometric signature of the patient, obtaining the biometric signature of the patient by scanning a bodily feature of the patient, decrypting the encrypted private key using the biometric signature of the patient to determine a private key associated with the patient, and accessing the medical records of the patient, using a combination of the public key and the private key associated with the patient, to access a local storage medium of the wearable device.

### RELATED APPLICATIONS

This application claims the benefit of U.S. Provisional Patent No. 62/433,956, filed Dec. 14, 2016, entitled “Obtaining a Medical Record Stored on a Blockchain from a Wearable Device,” the contents of which are incorporated by reference herein in their entirety.

### FIELD OF TECHNOLOGY

The following relates to obtaining medical records stored on a blockchain, and more specifically to a method and system for obtaining a medical record stored on the blockchain for a patient from a wearable device.

### BACKGROUND

Storing records on a blockchain is appealing due to the difficulty in modifying records and data already stored on the blockchain. A private key and a public key are needed to access a particular block or blockchain data, and the private key which is unique to a user is needed to generate a new transaction on the blockchain. If medical records were stored on the blockchain, the private key of the patient would be needed to view/modify the records. In the case where a user is unconscious or incapacitated in an emergency, a first responder could not access the user's medical records stored on the blockchain.

Thus, there is a need for a method and system for obtaining a medical record stored on the blockchain when the owner of the private key cannot readily provide the private key.

### SUMMARY

A first aspect relates to a method for obtaining a medical record of a patient that is unable to communicate, wherein the medical record of the patient is stored on a blockchain, comprising: receiving, by a processor of a computing system, an encrypted private key and a public key associated with the patient stored on a wearable device of the patient, in response to a scanning of the wearable device of the patient at a scene of an emergency, wherein the encrypted private key is decrypted by a biometric signature of the patient, obtaining, by the processor, the biometric signature of the patient by scanning a bodily feature of the patient, decrypting, by the processor, the encrypted private key using the biometric signature of the patient to determine a private key associated with the patient, and accessing, by the processor, the medical records of the patient, using a combination of the public key and the private key associated with the patient, to access a local storage medium of the wearable device.

A second aspect relates to a computer system, comprising: a processor, a biometric scanner coupled to the processor, a memory device coupled to the processor, and a computer readable storage device coupled to the processor, wherein the storage device contains program code executable by the processor via the memory device to implement a method for obtaining a medical record of a patient that is unable to communicate, wherein the medical record of the patient is stored on a blockchain, the method comprising: receiving, by a processor of a computing system, an encrypted private key and a public key associated with the patient stored on a wearable device of the patient, in response to a scanning of the wearable device of the patient at a scene of an emergency, wherein the encrypted private key is decrypted by a biometric signature of the patient, obtaining, by the processor, the biometric signature of the patient by scanning a bodily feature of the patient, decrypting, by the processor, the encrypted private key using the biometric signature of the patient to determine a private key associated with the patient, and accessing, by the processor, the medical records of the patient, using a combination of the public key and the private key associated with the patient, to access a local storage medium of the wearable device.

A third aspect relates to a computer program product, comprising a computer readable hardware storage device storing a computer readable program code, the computer readable program code comprising an algorithm that when executed by a computer processor of a computing system implements a method for obtaining a medical record of a patient that is unable to communicate, wherein the medical record of the patient is stored on a blockchain, comprising: receiving, by a processor of a computing system, an encrypted private key and a public key associated with the patient stored on a wearable device of the patient, in response to a scanning of the wearable device of the patient at a scene of an emergency, wherein the encrypted private key is decrypted by a biometric signature of the patient, obtaining, by the processor, the biometric signature of the patient by scanning a bodily feature of the patient, decrypting, by the processor, the encrypted private key using the biometric signature of the patient to determine a private key associated with the patient, and accessing, by the processor, the medical records of the patient, using a combination of the public key and the private key associated with the patient, to access a local storage medium of the wearable device.

The foregoing and other features of construction and operation will be more readily understood and fully appreciated from the following detailed disclosure, taken in conjunction with accompanying drawings.

### BRIEF DESCRIPTION OF THE DRAWINGS

Some of the embodiments will be described in detail, with reference to the following figures, wherein like designations denote like members, wherein:

FIG. 1 depicts a block diagram of a medical record accessing system, in accordance with embodiments of the present invention;

FIG. 2 depicts a block diagram of a wearable device, in accordance with embodiments of the present invention

FIG. 3 depicts an embodiment of a publicly distributable transactions ledger, in accordance with embodiments of the present invention;

FIG. 4 depicts a blockchain and two exemplary blocks of the blockchain, in accordance with embodiments of the present invention.

FIG. 5 depicts a block diagram of a first responder device 411, in accordance with embodiments of the present invention.

FIG. 6 depicts a flow chart of a method for obtaining a medical record stored on the blockchain for a patient from a wearable device, in accordance with embodiments of the present invention; and

FIG. 7 illustrates a block diagram of a computer system for the medical record accessing system of FIG. 1, capable of implementing methods for obtaining a medical record stored on the blockchain for a patient from a wearable device of FIG. 5, in accordance with embodiments of the present invention.

### DETAILED DESCRIPTION

Although certain embodiments are shown and described in detail, it should be understood that various changes and modifications may be made without departing from the scope of the appended claims. The scope of the present disclosure will in no way be limited to the number of constituting components, the materials thereof, the shapes thereof, the relative arrangement thereof, etc., and are disclosed simply as an example of embodiments of the present disclosure. A more complete understanding of the present embodiments and advantages thereof may be acquired by referring to the following description taken in conjunction with the accompanying drawings, in which like reference numbers indicate like features.

As a preface to the detailed description, it should be noted that, as used in this specification and the appended claims, the singular forms “a”, “an” and “the” include plural referents, unless the context clearly dictates otherwise.

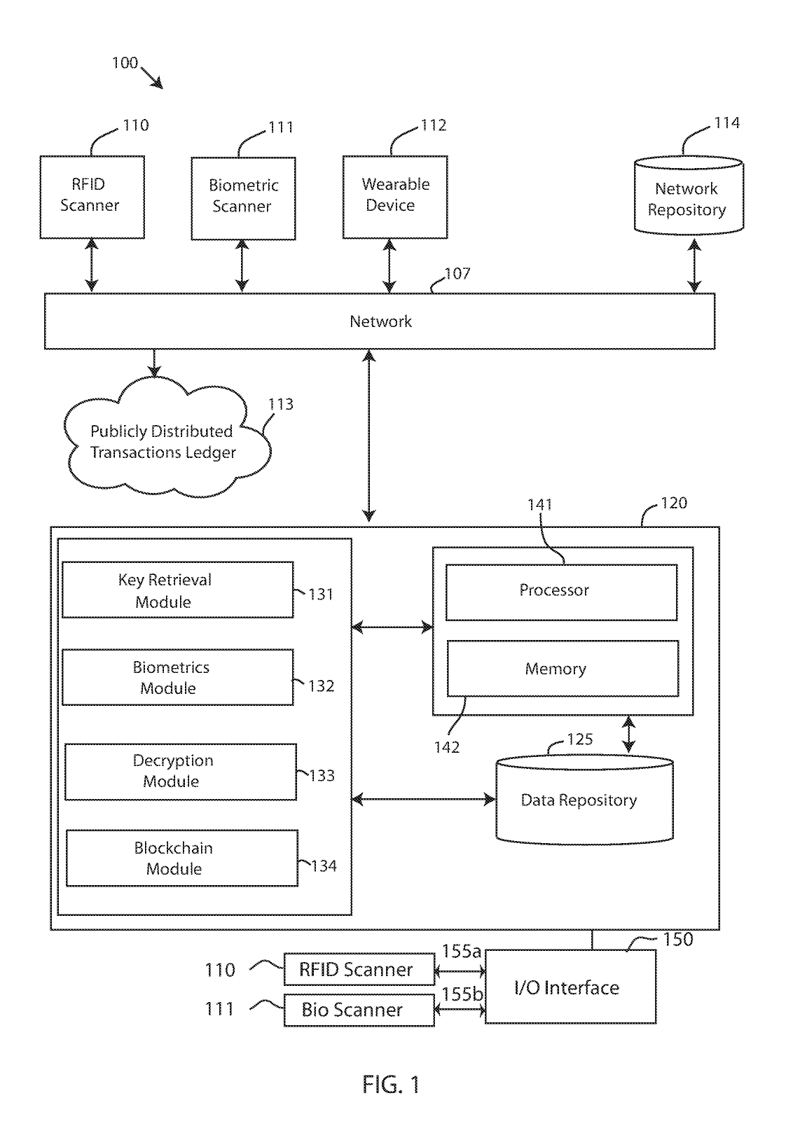

Referring to the drawings, FIG. 1 depicts a block diagram of a medical record accessing system 100, in accordance with embodiments of the present invention. Embodiments of a medical record accessing system 100 may be described as a system for obtaining, acquiring, accessing, securing, viewing, procuring, a medical record or medical history of a patient, wherein the medical record/history is stored on the blockchain. Embodiments of medical record accessing system 100 may comprise a RFID scanner 110 and a biometric scanner 111 communicatively coupled to the computing system 120 over via an I/O interface 150 and/or over a network 107. For instance, the RFID scanner 110 and the biometric scanner 111 may be connected via an I/O interface 150 to computer system 120 via data bus lines 155a, 155b (referred to collectively as “data bus lines 155) and/or over network 107. As shown in FIG. 1, the RFID scanner 110 and biometric scanner 111 may transmit information/data to the computing system 120. For example, the RFID scanner 110 may scan a wearable device 112 worn by a person who is unconscious or incapacitated at a scene of an emergency, and transmit the scanned data retrieved from the wearable device 112 to the computing system 120 via the data bus lines 155 to an I/O interface 150. Embodiments of the biometric scanner 111 may scan a bodily feature of a person who is unconscious or incapacitated at a scene of an emergency, and transmit the scanned biometric data retrieved from the person to the computing system 120 via the data bus lines 155 to the I/O interface 150. An I/O interface 150 may refer to any communication process performed between the computer system 120 and the environment outside of the computer system 120, for example, the sensors 110. Input to the computing system 120 may refer to the signals or instructions sent to the computing system 120, for example the data collected by the RFID scanner 110 and/or biometric scanner 111, while output may refer to the signals sent out from the computer system 120.

Alternatively, the RFID scanner 110 may scan a wearable device 112 worn by a person who is unconscious or incapacitated at a scene of an emergency, and transmit the scanned data retrieved from the wearable device 112 to the computing system 120 over network 107. Embodiments of the biometric scanner 111 may scan a bodily feature of a person who is unconscious or incapacitated at a scene of an emergency, and transmit the scanned biometric data retrieved from the person to the computing system 120 over network 107. A network 107 may refer to a group of two or more computer systems linked together. Network 107 may be any type of computer network known by individuals skilled in the art. Examples of computer networks 107 may include a LAN, WAN, campus area networks (CAN), home area networks (HAN), metropolitan area networks (MAN), an enterprise network, cloud computing network (either physical or virtual) e.g. the Internet, a cellular communication network such as GSM or CDMA network or a mobile communications data network. The architecture of the network 107 may be a peer-to-peer network in some embodiments, wherein in other embodiments, the network 107 may be organized as a client/server architecture.

In some embodiments, the network 107 may further comprise, in addition to the computing system 120, RFID scanner 110, biometric scanner 111, and wearable device 112, a connection to one or more network accessible knowledge bases containing information of one or more users, network repositories 114 or other systems connected to the network 107 that may be considered nodes of the network 107. In some embodiments, where the network repositories 114 allocate resources to be used by the other nodes of the network 107, the computing system 120 and network repository 114 may be referred to as servers.

The network repository 114 may be a data collection area on the network 107 which may back up and save all the data transmitted back and forth between the nodes of the network 107. For example, the network repository 114 may be a data center saving and cataloging data regarding medical treatment to generate both historical and predictive reports regarding a particular user. In some embodiments, a data collection center housing the network repository 114 may include an analytic module capable of analyzing each piece of data being stored by the network repository 114. Further, the computing system 120 may be integrated with or as a part of the data collection center housing the network repository 114. In some alternative embodiments, the network repository 114 may be a local repository (not shown) that is connected to the computing system 120.

Referring still to FIG. 1, embodiments of the computing system 120 may receive scanned data and other information from the RFID scanner 110 and the biometric scanner 111 which may be used by a first responder at a scene of emergency. Embodiments of the RFID scanner 110 may be a scanning device or other input mechanism that may scan, read, analyze, or otherwise retrieve information from a RFID chip or other chip located within the wearable device 112. The RFID scanner 110 may have a transmitter for transmitting scanned information to the computing system 120. Embodiments of the RFID scanner may be a handheld device operated by a first responder. Embodiments of the biometric scanner 111 may be a biometric scanner or reader or other input mechanism that may scan, read, analyze, or otherwise retrieve a biometric signature of a person. The biometric scanner may have a transmitter for transmitting scanned biometric information to the computing system 120. Embodiments of the biometric scanner 111 may be a fingerprint sensor, a facial recognition sensor, a retina scanner, an iris scanner, a blood/DNA analyzer, or other sensor or input device that may collect, capture, scan, or retrieve a biometric signal from a bodily feature of a person.

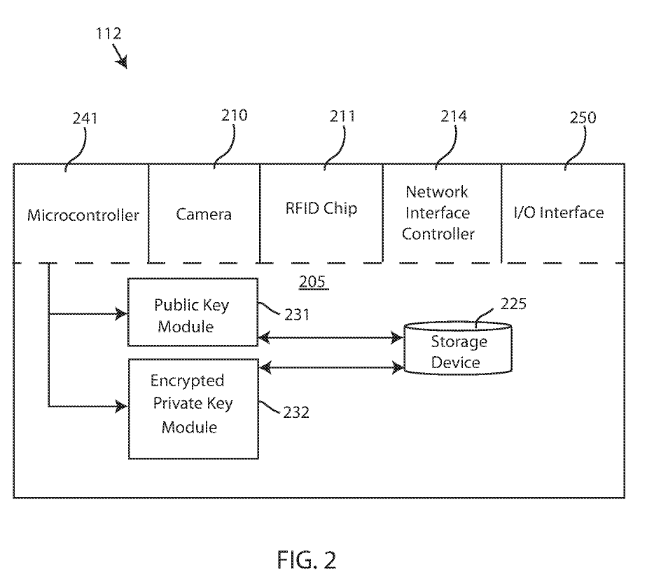

FIG. 2 depicts a block diagram of a wearable device 112, in accordance with embodiments of the present invention. Embodiments of the wearable device 112 may be configured to be worn or otherwise possessed by a person. Embodiments of the wearable device 112 may be a bracelet, a wearable computing device, a ring, an accessory, a necklace, and the like. The wearable device 112 may include a housing or enclosure that may house, protect, or otherwise comprise one or hardware components such as a processor or microcontroller 241, camera 210, RFID chip 211, network interface controller 214, and I/O interface 250. Software components of the wearable device 112 may be located in a memory system 205 of the wearable device 112. Embodiments of the wearable device 112 may include a microcontroller 241 for implementing the tasks associated with the wearable device 112. The RFID chip 211 may include various information that may be communicated to the RFID scanner 110 and ultimately to computing system 120. In some embodiments, the wearable device 112 may be comprised of the RFID chip 211 implanted into a skin of the user, wherein the RFID chip 211 includes the private key and the encrypted private key that may be communicated to the computing system 120. Further, embodiments of the wearable device 112 may include a camera 210 to perform a verifying task that the person operating computing system 120 is indeed an authority of first responder. For example, the wearable device 112 may require that a first responder or other authority show identification, wherein the camera 210 may capture an image of the identification for processing by the wearable device 112.

Embodiments of the network interface controller 214 may be a hardware component of the wearable device 112 that may connect the wearable device 112 to network 107. The network interface controller may transmit and receive data, including the transmission of data stored on the wearable device 112. In some embodiments, the data, such as a public key and an encrypted private key, may be stored in storage device 225 of memory system 205 of the wearable device 112. The network interface controller 214 may access the storage device 225, and transmit the data over the network 107 to the computing system 120. Alternatively, the medical records of the patient may be stored directly on the wearable device 112, such as a flash memory drive or solid state drive of the wearable device 112. Thus, once the private key and the public key are determined, the combination of the keys may be used to access storage 225 of the wearable device 112. By accessing the local storage device 225 of the wearable device 112 directly, the process for accessing the records becomes more computationally efficient by avoiding the need to access, view, and/or download the medical records over the cloud. Further, the storage device 225 of the wearable device may only store essential, vital, and/or emergency-specific medical data to limit the need for a large storage capacity of the wearable device to reduce costs and computational complexity in an emergency situation.

Additionally, embodiments of wearable device 112 may include an I/O interface 250. An I/O interface 250 may refer to any communication process performed between the wearable device 112 and the environment outside of the wearable device 112.

Furthermore, embodiments of the memory system 205 of the wearable device 112 may include a public key module 231 and an encrypted private key module 232. A “module” may refer to a hardware based module, software based module or a module may be a combination of hardware and software. Embodiments of hardware based modules may include self-contained components such as chipsets, specialized circuitry and one or more memory devices, while a software-based module may be part of a program code or linked to the program code containing specific programmed instructions, which may be loaded in the memory system 205 of the wearable device 112. A module (whether hardware, software, or a combination thereof) may be designed to implement or execute one or more particular functions or routines.

Embodiments of the public key module 231 may include one or more components of hardware and/or software program code for retrieving the public key associated with the patient/user's medical record/history stored on the blockchain. Moreover, embodiments of the wearable device 112 may include an encrypted private key module 232. Embodiments of the encrypted private key module 232 may include one or more components of hardware and/or software program code for generating, retrieving, and/or providing an encrypted private key for use with the public key to access medical records/history of the user stored on the blockchain or the storage device 225 of the wearable device 112. In an alternative embodiment, a blockchain database may be stored on the wearable device 112, such that the vital medical records may be stored locally on the wearable device 112, but also take advantage of the blockchain's immutable characteristics.

Referring back to FIG. 1, embodiments of the computing system 120 may include a key retrieval module 131, a biometrics module 132, a decryption module 133, and blockchain module 134. A “module” may refer to a hardware based module, software based module or a module may be a combination of hardware and software. Embodiments of hardware based modules may include self-contained components such as chipsets, specialized circuitry and one or more memory devices, while a software-based module may be part of a program code or linked to the program code containing specific programmed instructions, which may be loaded in the memory device of the computing system 120. A module (whether hardware, software, or a combination thereof) may be designed to implement or execute one or more particular functions or routines.

Embodiments of the key retrieval module 131 may include one or more components of hardware and/or software program code for retrieving, obtaining, or otherwise receiving or processing a public key and an encrypted private key from the wearable device 112. For instance, the key retrieval module 131 may receive a public key from the RFID scanner 110, in response to the RFID scanner 110 scanning the wearable device. When the wearable device 112 is scanned by the RFID scanner 110, the wearable device may offer a public key to be used by the computing system 120 to access the blockchain. In an exemplary embodiment, a first responder may arrive to a scene of an emergency, wherein a person is unconscious, incapacitated, in shock, or otherwise unable to communicate with the first responder, and may use the RFID scanner 110 to scan the wearable device 112. The wearable device 112 may communicate a public key to the RFID scanner 110, which then may be transmitted to the computing system 120. The key retrieval module 131 may store the public key for later use with the blockchain. Furthermore, embodiments of the key retrieval module 131 may receive an encrypted private key from the RFID scanner. For instance, when the RFID scanner 110 scans the wearable device 112, the wearable device 112 may offer, in addition to the public key, an encrypted private key, which may then be transmitted to the computing system 120. The key retrieval module 131 may store the encrypted private key for later use with the blockchain. Embodiments of the encrypted private key may be a private key associated with the user/patient/person being treated at the scene, which is encrypted. The encrypted private key may be unique to the user for accessing the person's medical records stored on the blockchain. In an exemplary embodiment, the encrypted private key may be decrypted by a biometrical signature of the patient.

Embodiments of the computing system 120 may also include a biometrics module 132. Embodiments of the biometrics module 132 may include one or more components of hardware and/or software program code for receiving and/or obtaining a biometric signature of the person/patient to decrypt the encrypted private key. For instance, the biometrics module 132 may receive a biometric signature from the biometric scanner 111, in response to the biometric scanner 111 scanning a bodily feature of the patient. Embodiments of the bodily feature may include a face, a finger, a thumb, an eye, an iris, a retina, a blood composition, a skin or tissue, and the like. The biometric signature captured by the biometric scanner may be transmitted to the computing system 120.

Referring still to FIG. 1, embodiments of the computing system 120 may include a decryption module 133. Embodiments of the decryption module 133 may include one or more components of hardware and/or software program code for decrypting the encrypted private key using the biometric signature. For instance, the decryption module 133 may use the metadata and/or data contained in the biometric signature of the patient to computationally decrypt the encrypted private key, to obtain the private key associated with the patient/user/person being treated at the scene.

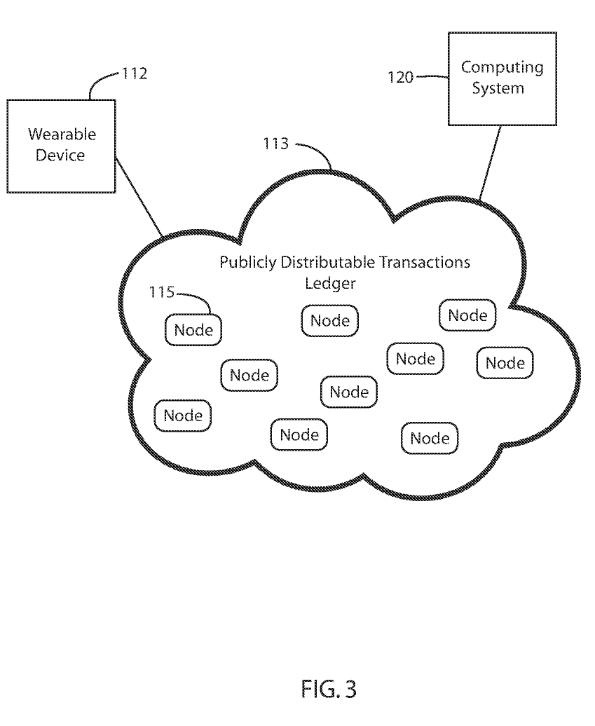

Embodiments of the computing system 120 may further include a blockchain module 133. Embodiments of the blockchain module 133 may include one or more components of hardware and/or software program code for accessing the publicly distributed transactions ledger 113 (i.e. blockchain) to view a medical record or medical history of the patient, using the public key and the private key received by the computing system 120. Medical records may be recorded on the publicly distributable transactions ledger 113. The recordation of the medical records is immutable and almost impossible to fraudulently change the details of the records stored on the ledger 113 due to the nature of the decentralized ledger, otherwise referred to as the blockchain. FIG. 3 depicts an embodiment of a publicly distributable transactions ledger 113, in accordance with embodiments of the present invention. Embodiments of ledger 113 may be a distributed peer-to-peer network, including a plurality of nodes 115. The ledger 113 may represent a computing environment for operating a decentralized framework that can maintain a distributed data structure. In other words, ledger 113 may be a secure distributed transaction ledger or a blockchain that may support document management. Each node 115 may maintain an individual public ledger (i.e. maintained publicly) according to set procedures that employ cryptographic methods and a proof-of-work concept. In view of the public nature of the ledger and the proof-of-work concept, the nodes 115 collectively create a decentralized, trusted network. Further, embodiments of the publicly decentralized trusted ledger 113 may be accessible by the computing system 120 and the wearable device 112 for verifying a transaction, completing a transaction, or viewing a medical record.

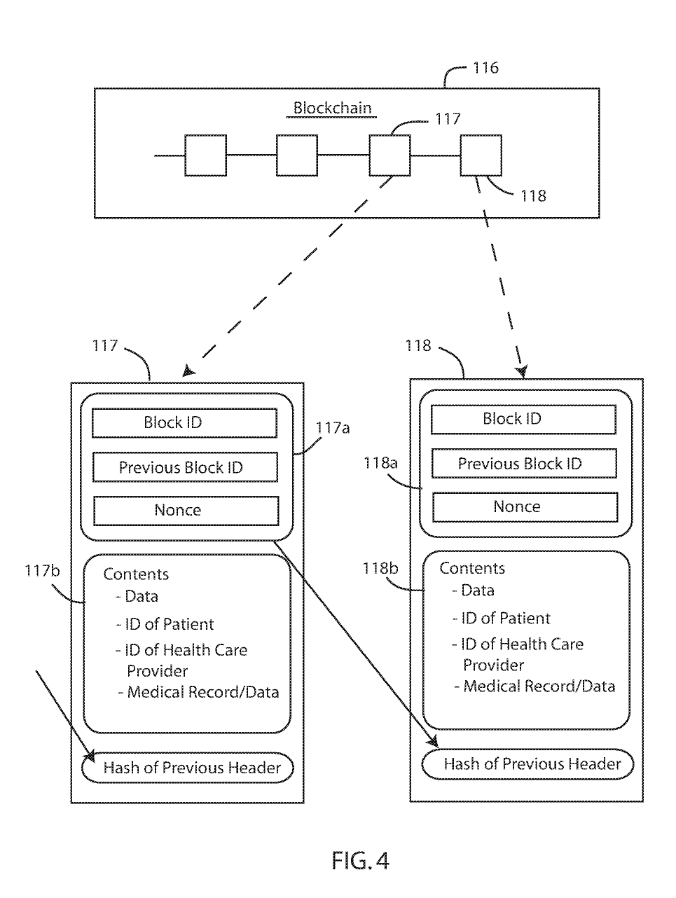

FIG. 4 depicts a blockchain 116 and two exemplary blocks 117, 118 of the blockchain 116, in accordance with embodiments of the present invention. Embodiments of the blockchain 116 may represent the publicly distributable transactions ledger 113, and may include a plurality of blocks. Each block, such as block 117 and block 118 may include data regarding recent transactions and/or contents relating to medical records of a patient, linking data that links one block 118 to a previous block 117 in the blockchain, proof-of-work data that ensures that the state of the blockchain 116 is valid, and is endorsed/verified by a majority of the record keeping system. The confirmed transactions of the blockchain are done using cryptography to ensure that the integrity and the chronological order of the blockchain are enforced and can be independently verified by each node 115 of the blockchain 116. New transactions may be added to the blockchain 116 using a distributed consensus system that confirms pending transactions using a mining process, which means that each transaction can easily be verified for accuracy, but very difficult or impossible to modify. Moreover, embodiments of a block 117 of the blockchain 116 may include a header 117a and a content 117b. Embodiments of the header 117a may include a block ID, a previous block ID, and a nonce. The nonce may represent a proof-of-work. The header 117a may be used to link block 117 to other blocks of the blockchain. Embodiments of the block contents 117b may include transaction information relating to a transaction for adding a new medical record. Likewise, block 118 may include a header 118a and contents 118b. Block 118 includes a hash of the previous block's header (i.e. 117a), thereby linking the blocks 117, 118 to the blockchain.

The transaction information cannot be modified without at least one of the nodes 115 noticing; thus, the blockchain 116 can be trusted to verify transactions occurring on the blockchain 116. Further, access to blocks of a blockchain 116 that include private medical records may only be accessible using a public key associated with the patient's account or identity, which is publicly available, along with a private key unique to the patient, and known only to the patient.

Accordingly, embodiments of the blockchain module 134 shown in FIG. 1 may use the public key and the private key received by the computing system 120 to gain access to the medical records of the patient that are stored on the blockchain 116, or on a local storage medium 225 of the wearable device 112. The patient's medical records may then be viewed by a first responder or other authority at the scene of an emergency when the patient cannot communicate the patient's private key to the first responder or authority present on the scene. Furthermore, embodiments of the blockchain module 134 may generate a new transaction on the blockchain using the private key, the new transaction being that the medical record of the unconscious patient was retrieved at the scene of the emergency, as well as additional data that belongs in the medical record.

Embodiments of the computing system 120 may be equipped with a memory device 142 which may store various information and data regarding the scanned data, and a processor 141 for implementing the tasks associated with the medical record accessing system 100.

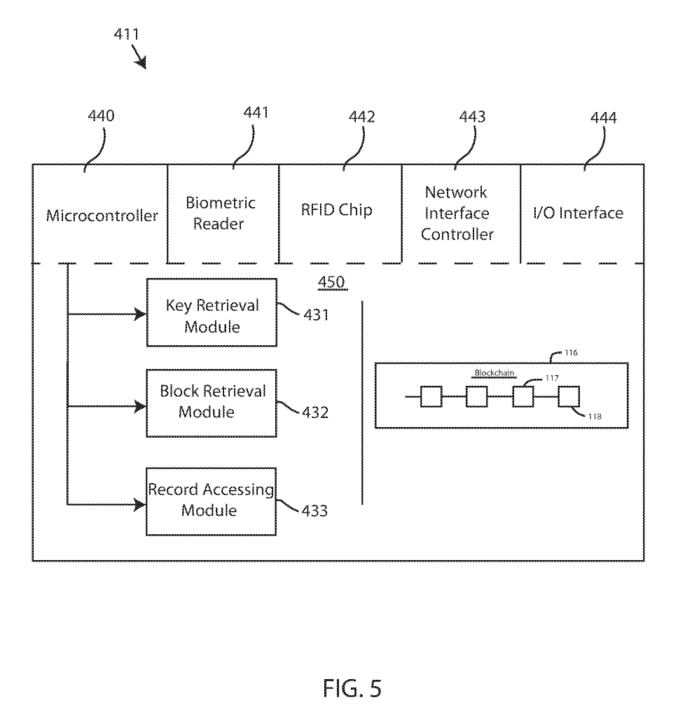

With reference now to FIG. 5, which depicts a block diagram of a first responder device 411, in accordance with embodiments of the present invention. Embodiments of the first responder device 412 may be a medical personnel device, an authorized medical care provider device, a computing device, mobile device, etc., that may be possessed, worn, and/or carried by a first responder, doctor, nurse, surgeon, medical care provider, or other medical personnel. Embodiments of the first responder device 411 may be a scanner, a mobile computer, a smartphone, a handheld device, a portable terminal, and the like. The first responder device 411 may include a housing or enclosure that may house, protect, or otherwise comprise one or hardware components such as a processor or microcontroller 440, biometric reader 441, RFID scanner 442, network interface controller 443, and I/O interface 444. Software components of the first responder device 411 may be located in a memory system 450 of the first responder device 411. Embodiments of the first responder device 411 may include a microcontroller 440 for implementing the tasks associated with the first responder device 411. The biometric reader 441 may be one or more sensors, readers, input mechanisms, and the like to capture a biometric marker of an individual. Embodiments of the biometric reader 441 may be a facial recognition sensor, a thumbprint reader, a camera, an infrared sensor, a microphone, and/or a combination thereof. Embodiments of the first responder device 411 may further include a RFID chip 442 for communicating with the wearable device 112 to obtain a private key stored on the wearable device 112. Further, embodiments of the first responder device 411 a network interface controller 443, which may be a hardware component of the first responder device 411 that may connect the first responder device 411 to a network. The network interface controller 443 may transmit and receive data, including the transmission of data stored on the first responder device 411, and to post new transactions to the blockchain in accordance with a consensus algorithm, such that the blockchain 16 may be updated as necessary. Additionally, embodiments of first responder device 411 may include an I/O interface 250. An I/O interface 250 may refer to any communication process performed between the first responder device 411 and the environment outside of the first responder device 411.

Embodiments of the first responder device 411 (and/or the wearable device 112) may include a blockchain 116 storage medium locally on the first responder device 411. The blockchain 116 may be a decentralized database for storing medical records for a plurality of individuals, wherein each first responder device 411 belonging to a first responder or other trusted medical personnel contains the blockchain database 116. The blockchain 116 may contain medical records for individuals, but a particular block of the blockchain 116 containing a medical record associated with an individual may not be accessible without a private key stored on the wearable device 112, worn by the individual. Once the private key is obtained from the wearable device 112, using, for example, NFC technology to retrieve the private key, the private key may be used to fetch the block containing the individual's medical records from the plurality of blocks 117, 118 of the blockchain 116. The block fetched by the private key may still be unreadable/encrypted, which may be decrypted by a successful match between a biometric signature data stored on the blockchain 116 and a biometric signature captured by the first responder device 411 using the biometric reader 441. The successful match between the stored and the captured biometric signature may decrypt the block associated with the individual stored locally on the first responder device 411, allowing the first responder to view and/or access the medical record of the individual. In some embodiments, only a subset of the block may be decrypted, wherein the subset only reveals essential, vital, and/or emergency related information, such as blood type, drug allergies, medications being taken, and the like. Accordingly, accessing a medical record in a secure and protected manner can be accomplished in a more computationally efficient manner in an emergency situation by utilizing the blockchain's immutable characteristics but avoiding a need to connect to the cloud to access medical records.

Embodiments of the memory system 450 of the first responder device 411 may include a key retrieval module 431, a block retrieval module 432, and a record accessing module 433. A “module” may refer to a hardware based module, software based module or a module may be a combination of hardware and software. Embodiments of hardware based modules may include self-contained components such as chipsets, specialized circuitry and one or more memory devices, while a software-based module may be part of a program code or linked to the program code containing specific programmed instructions, which may be loaded in the memory system 450 of the first responder device 411. A module (whether hardware, software, or a combination thereof) may be designed to implement or execute one or more particular functions or routines.

Embodiments of the key retrieval module 431 may include one or more components of hardware and/or software program code for retrieving the private key stored locally on the wearable device 112. Embodiments of the key retrieval module 431 of the first responder device 411 may initiate a communication with the wearable device 112 using a short range communication technology, such as NFC, Bluetooth®, RF, and the like, to retrieve, obtain, acquire, or otherwise receive the private key from wearable device 112. Embodiments of the first responder device 411 may also include block retrieval module 432. Embodiments of the block retrieval module 432 may include one or more components of hardware and/or software program code for retrieving the block associated with an individual (e.g. person unconscious at a scene of emergency) from the blockchain 16 database locally stored on the first responder device 411. For instance, embodiments of the block retrieval module 432 may use the private key to search for a particular block in the blockchain 16 that contains medical information of the individual wearing the wearable device 112. The private key may contain identifying information that may also be contained in the block of the blockchain 16 for searching, locating, and fetching the block, such as an identification number of the block in the blockchain, which may be completely anonymous to the individual. The block and/or contents of the block fetched by the block retrieval module 432 may be encrypted or otherwise unreadable to the person or computer when the block is fetched. In an exemplary embodiment, the block fetched using the private key may remain encrypted even after the private key is obtained from the wearable device 112 because it is possible that the individual wearing the wearable device 112 is not actually the person associated with the wearable device 112. In other words, if the person wearing the wearable device 112 is not the individual that owns the wearable device 112, a first responder may apply incorrect, vital medical information to an emergency situation.

Thus, embodiments of the first responder device 411 may include a record accessing module 433. Embodiments of the record accessing module 433 may include one or more components of hardware and/or software program code for authenticating/confirming an identity of the individual for accessing the medical records stored locally on the first responder device 411. In particular, the record access module 433 may initiate a real-time capture of a biometric marker of the individual, using the biometric reader 441 of the first responder device 411. The first responder device 411 may capture, scan, collect, or otherwise receive or obtain a biometric signature, such as data resulting from a facial scan, a retinal data, a thumbprint scan, and the like. The real-time data from the biometric signature may be compared to a biometric signature stored on the block, and if the comparison results in a successful match, the block may be decrypted. For example, the block fetched using the private key may remain encrypted, except for meta data relating to a biometric signature stored on the block, which cannot be changed without being noticed by the blockchain. If the real-time capture of the biometric signal matches the stored biometric signal, an identity may be confirmed that the medical record information is indeed associated with the individual, and the record access module 433 may access, retrieve, obtain, or otherwise provide medical records and information from the local database of the first responder device 411, for use in providing medical care. In an exemplary embodiment, to further maintain security and privacy, the block may only be decrypted to allow access to a subset of the block, wherein the subset of the block may contain only the most essential and vital medical information. Further, the access may be time-sensitive, such as good only a single time, or for a limited amount of time.

Additionally, the medical record/information of the patient, once obtained may cascade to other medical care entities, such as a surgeon, an emergency room, a hospital, an ambulance, and the like. For instance, the device 411may transmit the medical information over traditional communication pathways to internet connected devices controlled by the other medical care entities. Thus, as the patient is transported from one medical care entity to another medical care entity, the vital medical records and medical information can be known to the medical care personnel prior to the patient arriving, saving time by not having to access the patient's wearable device 112. In alternative embodiments, the first responder device 411 may post a new transaction to the blockchain that may allow verified sources to access the medical records/information of the patient from the blockchain, without needing the biometric authentication. The access may be for a limited time, or may be triggered by a physical location of the wearable device 112. For example, access settings to the newly posted medical records on the blockchain may be accessible as the wearable device 112 is geolocated within a predetermined location or within a certain proximity of the medical care entity computing device.

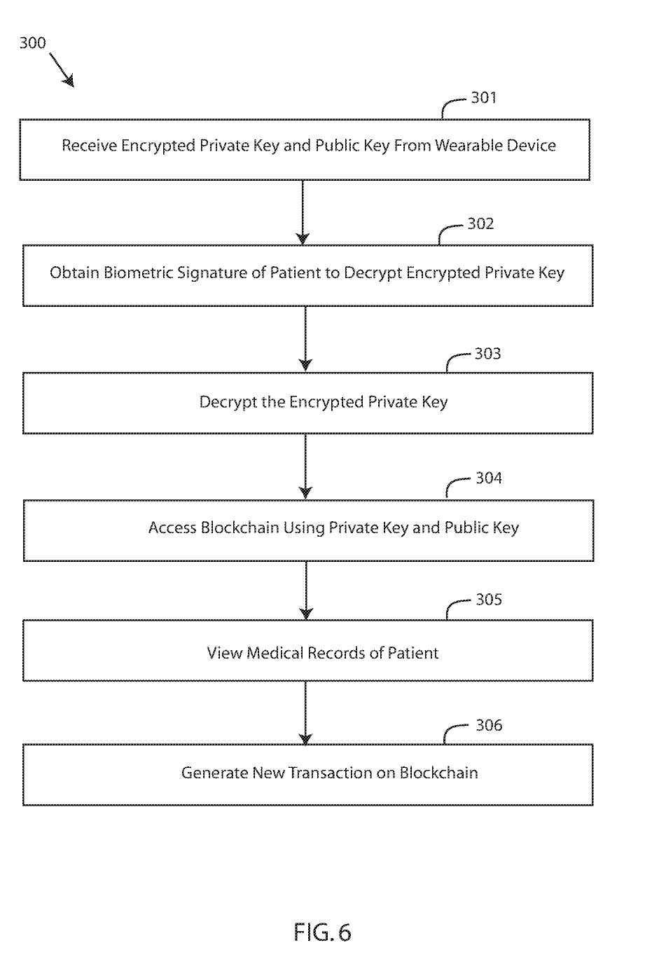

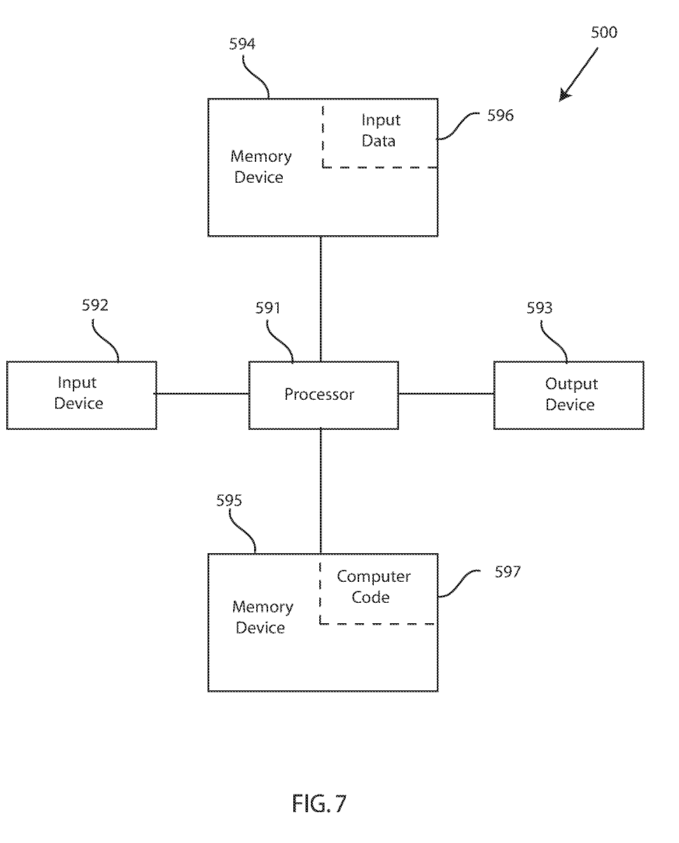

Referring now to FIG. 6, which depicts a flow chart of a method 300 obtaining a medical record stored on the blockchain for a patient from a wearable device, in accordance with embodiments of the present invention. One embodiment of a method 300 or algorithm that may be implemented obtaining a medical record stored on the blockchain for a patient from a wearable device in accordance with the medical record accessing system 100 described in FIG. 1 using one or more computer systems as defined generically in FIG. 7 below, and more specifically by the specific embodiments of FIGS. 1-5.

Embodiments of the method 300 for obtaining a medical record stored on the blockchain for a patient from a wearable device may begin at step 301 wherein a public key and an encrypted private key are received by the computing system 120 from the wearable device 112. The private key and the encrypted private key may be received by the computing system 120, in response to the wearable device being scanned by the RFID scanner 110. Step 302 obtains or receives a biometric signature of the patient in possession of the wearable device 112. The biometric signature may be required to decrypt the encrypted private key. Step 303 decrypts the encrypted private key using the received biometric signature of the patient, so that the private key is known to the computing system 120. Step 304 accesses the part of the blockchain 116 that contains the patient's medical records, using a combination of the public key and the private key, received from the wearable device 112 worn or otherwise possessed by the patient. Step 305 views the medical records and presents, displays, or otherwise provides the medical records of the patient to the first responder so that appropriate medical and treatment decisions can be made during treatment of the patient. Step 306 generates a new transaction on the blockchain that the private key has been used to access the medical records. Additionally, a new transaction may be generated if medical treatment is received by the patient.

FIG. 7 illustrates a block diagram of a computer system for the medical record accessing system of FIG. 1, capable of implementing methods for obtaining a medical record stored on the blockchain for a patient from a wearable device of FIG. 6, in accordance with embodiments of the present invention. The computer system 500 may generally comprise a processor 591, an input device 592 coupled to the processor 591, an output device 593 coupled to the processor 591, and memory devices 594 and 595 each coupled to the processor 591. The input device 592, output device 593 and memory devices 594, 595 may each be coupled to the processor 591 via a bus. Processor 591 may perform computations and control the functions of computer 500, including executing instructions included in the computer code 597 for the tools and programs capable of implementing a method for obtaining a medical record stored on the blockchain for a patient from a wearable device, in the manner prescribed by the embodiments of FIG. 5 using the medical record accessing system of FIG. 1, wherein the instructions of the computer code 597 may be executed by processor 591 via memory device 595. The computer code 597 may include software or program instructions that may implement one or more algorithms for implementing the methods for obtaining a medical record stored on the blockchain for a patient from a wearable device, as described in detail above. The processor 591 executes the computer code 597. Processor 591 may include a single processing unit, or may be distributed across one or more processing units in one or more locations (e.g., on a client and server).

The memory device 594 may include input data 596. The input data 596 includes any inputs required by the computer code 597. The output device 593 displays output from the computer code 597. Either or both memory devices 594 and 595 may be used as a computer usable storage medium (or program storage device) having a computer readable program embodied therein and/or having other data stored therein, wherein the computer readable program comprises the computer code 597. Generally, a computer program product (or, alternatively, an article of manufacture) of the computer system 500 may comprise said computer usable storage medium (or said program storage device).

Memory devices 594, 595 include any known computer readable storage medium, including those described in detail below. In one embodiment, cache memory elements of memory devices 594, 595 may provide temporary storage of at least some program code (e.g., computer code 597) in order to reduce the number of times code must be retrieved from bulk storage while instructions of the computer code 597 are executed. Moreover, similar to processor 591, memory devices 594, 595 may reside at a single physical location, including one or more types of data storage, or be distributed across a plurality of physical systems in various forms. Further, memory devices 594, 595 can include data distributed across, for example, a local area network (LAN) or a wide area network (WAN). Further, memory devices 594, 595 may include an operating system (not shown) and may include other systems not shown in FIG. 7.

In some embodiments, the computer system 500 may further be coupled to an Input/output (I/O) interface and a computer data storage unit. An I/O interface may include any system for exchanging information to or from an input device 592 or output device 593. The input device 592 may be, inter alia, a keyboard, a mouse, etc. or in some embodiments the scanners 110, 111. The output device 593 may be, inter alia, a printer, a plotter, a display device (such as a computer screen), a magnetic tape, a removable hard disk, a floppy disk, etc. The memory devices 594 and 595 may be, inter alia, a hard disk, a floppy disk, a magnetic tape, an optical storage such as a compact disc (CD) or a digital video disc (DVD), a dynamic random access memory (DRAM), a read-only memory (ROM), etc. The bus may provide a communication link between each of the components in computer 500, and may include any type of transmission link, including electrical, optical, wireless, etc.

An I/O interface may allow computer system 500 to store information (e.g., data or program instructions such as program code 597) on and retrieve the information from computer data storage unit (not shown). Computer data storage unit includes a known computer-readable storage medium, which is described below. In one embodiment, computer data storage unit may be a non-volatile data storage device, such as a magnetic disk drive (i.e., hard disk drive) or an optical disc drive (e.g., a CD-ROM drive which receives a CD-ROM disk). In other embodiments, the data storage unit may include a knowledge base or data repository 125 as shown in FIG. 1.

As will be appreciated by one skilled in the art, in a first embodiment, the present invention may be a method; in a second embodiment, the present invention may be a system; and in a third embodiment, the present invention may be a computer program product. Any of the components of the embodiments of the present invention can be deployed, managed, serviced, etc. by a service provider that offers to deploy or integrate computing infrastructure with respect to medical record accessing systems and methods. Thus, an embodiment of the present invention discloses a process for supporting computer infrastructure, where the process includes providing at least one support service for at least one of integrating, hosting, maintaining and deploying computer-readable code (e.g., program code 597) in a computer system (e.g., computer 500) including one or more processor(s) 591, wherein the processor(s) carry out instructions contained in the computer code 597 causing the computer system to access medical records stored on the blockchain for a patient that cannot communicate at a scene of an emergency. Another embodiment discloses a process for supporting computer infrastructure, where the process includes integrating computer-readable program code into a computer system including a processor.

The step of integrating includes storing the program code in a computer-readable storage device of the computer system through use of the processor. The program code, upon being executed by the processor, implements a method for obtaining a medical record stored on the blockchain for a patient from a wearable device. Thus, the present invention discloses a process for supporting, deploying and/or integrating computer infrastructure, integrating, hosting, maintaining, and deploying computer-readable code into the computer system 500, wherein the code in combination with the computer system 500 is capable of performing a method for obtaining a medical record stored on the blockchain for a patient from a wearable device.

A computer program product of the present invention comprises one or more computer readable hardware storage devices having computer readable program code stored therein, said program code containing instructions executable by one or more processors of a computer system to implement the methods of the present invention.

A computer system of the present invention comprises one or more processors, one or more memories, and one or more computer readable hardware storage devices, said one or more hardware storage devices containing program code executable by the one or more processors via the one or more memories to implement the methods of the present invention.

The present invention may be a system, a method, and/or a computer program product at any possible technical detail level of integration. The computer program product may include a computer readable storage medium (or media) having computer readable program instructions thereon for causing a processor to carry out aspects of the present invention.

The computer readable storage medium can be a tangible device that can retain and store instructions for use by an instruction execution device. The computer readable storage medium may be, for example, but is not limited to, an electronic storage device, a magnetic storage device, an optical storage device, an electromagnetic storage device, a semiconductor storage device, or any suitable combination of the foregoing. A non-exhaustive list of more specific examples of the computer readable storage medium includes the following: a portable computer diskette, a hard disk, a random access memory (RAM), a read-only memory (ROM), an erasable programmable read-only memory (EPROM or Flash memory), a static random access memory (SRAM), a portable compact disc read-only memory (CD-ROM), a digital versatile disk (DVD), a memory stick, a floppy disk, a mechanically encoded device such as punch-cards or raised structures in a groove having instructions recorded thereon, and any suitable combination of the foregoing. A computer readable storage medium, as used herein, is not to be construed as being transitory signals per se, such as radio waves or other freely propagating electromagnetic waves, electromagnetic waves propagating through a waveguide or other transmission media (e.g., light pulses passing through a fiber-optic cable), or electrical signals transmitted through a wire.

Computer readable program instructions described herein can be downloaded to respective computing/processing devices from a computer readable storage medium or to an external computer or external storage device via a network, for example, the Internet, a local area network, a wide area network and/or a wireless network. The network may comprise copper transmission cables, optical transmission fibers, wireless transmission, routers, firewalls, switches, gateway computers and/or edge servers. A network adapter card or network interface in each computing/processing device receives computer readable program instructions from the network and forwards the computer readable program instructions for storage in a computer readable storage medium within the respective computing/processing device.

Computer readable program instructions for carrying out operations of the present invention may be assembler instructions, instruction-set-architecture (ISA) instructions, machine instructions, machine dependent instructions, microcode, firmware instructions, state-setting data, configuration data for integrated circuitry, or either source code or object code written in any combination of one or more programming languages, including an object oriented programming language such as Smalltalk, C++, or the like, and procedural programming languages, such as the “C” programming language or similar programming languages. The computer readable program instructions may execute entirely on the user's computer, partly on the user's computer, as a stand-alone software package, partly on the user's computer and partly on a remote computer or entirely on the remote computer or server. In the latter scenario, the remote computer may be connected to the user's computer through any type of network, including a local area network (LAN) or a wide area network (WAN), or the connection may be made to an external computer (for example, through the Internet using an Internet Service Provider). In some embodiments, electronic circuitry including, for example, programmable logic circuitry, field-programmable gate arrays (FPGA), or programmable logic arrays (PLA) may execute the computer readable program instructions by utilizing state information of the computer readable program instructions to personalize the electronic circuitry, in order to perform aspects of the present invention.

Aspects of the present invention are described herein with reference to flowchart illustrations and/or block diagrams of methods, apparatus (systems), and computer program products according to embodiments of the invention. It will be understood that each block of the flowchart illustrations and/or block diagrams, and combinations of blocks in the flowchart illustrations and/or block diagrams, can be implemented by computer readable program instructions.

These computer readable program instructions may be provided to a processor of a general purpose computer, special purpose computer, or other programmable data processing apparatus to produce a machine, such that the instructions, which execute via the processor of the computer or other programmable data processing apparatus, create means for implementing the functions/acts specified in the flowchart and/or block diagram block or blocks. These computer readable program instructions may also be stored in a computer readable storage medium that can direct a computer, a programmable data processing apparatus, and/or other devices to function in a particular manner, such that the computer readable storage medium having instructions stored therein comprises an article of manufacture including instructions which implement aspects of the function/act specified in the flowchart and/or block diagram block or blocks.

The computer readable program instructions may also be loaded onto a computer, other programmable data processing apparatus, or other device to cause a series of operational steps to be performed on the computer, other programmable apparatus or other device to produce a computer implemented process, such that the instructions which execute on the computer, other programmable apparatus, or other device implement the functions/acts specified in the flowchart and/or block diagram block or blocks.

The flowchart and block diagrams in the Figures illustrate the architecture, functionality, and operation of possible implementations of systems, methods, and computer program products according to various embodiments of the present invention. In this regard, each block in the flowchart or block diagrams may represent a module, segment, or portion of instructions, which comprises one or more executable instructions for implementing the specified logical function(s). In some alternative implementations, the functions noted in the blocks may occur out of the order noted in the Figures. For example, two blocks shown in succession may, in fact, be executed substantially concurrently, or the blocks may sometimes be executed in the reverse order, depending upon the functionality involved. It will also be noted that each block of the block diagrams and/or flowchart illustration, and combinations of blocks in the block diagrams and/or flowchart illustration, can be implemented by special purpose hardware-based systems that perform the specified functions or acts or carry out combinations of special purpose hardware and computer instructions.

While embodiments of the present invention have been described herein for purposes of illustration, many modifications and changes will become apparent to those skilled in the art. Accordingly, the appended claims are intended to encompass all such modifications and changes as fall within the true spirit and scope of this invention.

The descriptions of the various embodiments of the present invention have been presented for purposes of illustration, but are not intended to be exhaustive or limited to the embodiments disclosed. Many modifications and variations will be apparent to those of ordinary skill in the art without departing from the scope and spirit of the described embodiments. The terminology used herein was chosen to best explain the principles of the embodiments, the practical application or technical improvement over technologies found in the marketplace, or to enable others of ordinary skill in the art to understand the embodiments disclosed herein

1. A method for obtaining a medical record of a patient that is unable to communicate, wherein the medical record of the patient is stored on a blockchain, comprising:
receiving, by a processor of a computing system, an encrypted private key and a public key associated with the patient stored on a wearable device of the patient, in response to a scanning of the wearable device of the patient at a scene of an emergency, wherein the encrypted private key is decrypted by a biometric signature of the patient;
obtaining, by the processor, the biometric signature of the patient by scanning a bodily feature of the patient;
decrypting, by the processor, the encrypted private key using the biometric signature of the patient to determine a private key associated with the patient; and
accessing, by the processor, the medical records of the patient, using a combination of the public key and the private key associated with the patient, to access a local storage medium of the wearable device.

2. The method of claim 1, wherein the wearable device is embedded into a skin of the patient.

3. The method of claim 1, wherein the wearable device is a bracelet.

4. The method of claim 1, wherein the local storage medium is a blockchain database stored on the wearable device, and contains only vital, emergency-specific information of the patient.

5. The method of claim 1, further comprising a biometric scanner coupled to the processor for scanning the biometric signature of the patient, and a RFID scanner coupled to the processor for scanning the wearable device.

6. The method of claim 1, further comprising generating a transaction on the blockchain that the medical record of the patient was retrieved at the scene of the emergency.

7. The method of claim 1, wherein the bodily feature of the patient is at least one of a fingerprint, an iris, a retina, and a facial feature.

8. A computer system, comprising:
a processor;
a biometric scanner coupled to the processor;
a memory device coupled to the processor; and
a computer readable storage device coupled to the processor, wherein the storage device contains program code executable by the processor via the memory device to implement a method for obtaining a medical record of a patient that is unable to communicate, wherein the medical record of the patient is stored on a blockchain, the method comprising:
receiving, by a processor of a computing system, an encrypted private key and a public key associated with the patient stored on a wearable device of the patient, in response to a scanning of the wearable device of the patient at a scene of an emergency, wherein the encrypted private key is decrypted by a biometric signature of the patient;
obtaining, by the processor, the biometric signature of the patient by scanning a bodily feature of the patient;
decrypting, by the processor, the encrypted private key using the biometric signature of the patient to determine a private key associated with the patient; and
accessing, by the processor, the medical records of the patient, using a combination of the public key and the private key associated with the patient, to access a local storage medium of the wearable device.

9. The computer system of claim 8, wherein the wearable device is embedded into a skin of the unconscious patient.

10. The computer system of claim 8, wherein the wearable device is a bracelet.

11. The computer system of claim 8, wherein the local storage medium is a blockchain database stored on the wearable device, and contains only vital, emergency-specific information of the patient.

12. The computer system of claim 8, further comprising a RFID scanner coupled to the processor.

13. The computer system of claim 8, further comprising generating a transaction on the blockchain that the medical record of the patient was retrieved at the scene of the emergency.

14. The computer system of claim 8, wherein the bodily feature of the patient is at least one of a fingerprint, an iris, a retina, and a facial feature.

15. A computer program product, comprising a computer readable hardware storage device storing a computer readable program code, the computer readable program code comprising an algorithm that when executed by a computer processor of a computing system implements a method for obtaining a medical record of a patient that is unable to communicate, wherein the medical record of the patient is stored on a blockchain, comprising:
receiving, by a processor of a computing system, an encrypted private key and a public key associated with the patient stored on a wearable device of the patient, in response to a scanning of the wearable device of the patient at a scene of an emergency, wherein the encrypted private key is decrypted by a biometric signature of the patient;
obtaining, by the processor, the biometric signature of the patient by scanning a bodily feature of the patient;
decrypting, by the processor, the encrypted private key using the biometric signature of the patient to determine a private key associated with the patient; and
accessing, by the processor, the medical records of the patient, using a combination of the public key and the private key associated with the patient, to access a local storage medium of the wearable device.

16. The computer program product of claim 15, wherein the wearable device is embedded into a skin of the patient.

17. The computer program product of claim 15, wherein the wearable device is a bracelet.

18. The computer program product of claim 15, further comprising a biometric scanner coupled to the processor for scanning the biometric signature of the patient, and a RFID scanner coupled to the processor for scanning the wearable device.

19. The computer program product of claim 15, wherein the local storage medium is a blockchain database stored on the wearable device, and contains only vital, emergency-specific information of the patient

20. The computer program product of claim 15, wherein the bodily feature of the patient is at least one of a fingerprint, an iris, a retina, and a facial feature.
